(19) United States
(12) Patent Application Publication
(10) Pub. No.: US 2016/0058691 A1
(43) Pub. Date: Mar. 3, 2016

(54) ODOR AND BACTERIAL TREATMENT USING OZONATED UNSATURATED FAT

(71) Applicant: Todd Platzer, Kure Beach, NC (US)

(72) Inventor: Todd Platzer, Kure Beach, NC (US)

(21) Appl. No.: 14/836,858

(22) Filed: Aug. 26, 2015

Related U.S. Application Data
(60) Provisional application No. 62/042,048, filed on Aug. 26, 2014.

(51) Int. Cl. A61K 8/92; A61Q 15/00

(52) U.S. Cl. CPC A61K 8/922; A61Q 15/00; A61K 8/927; A61K 2800/30

(57) ABSTRACT

A substance to treat body odor or bacteria is manufactured by providing a supply of ozone and providing unsaturated fat. The ozone is introduced into the unsaturated fat to create an ozonated unsaturated fat substance to treat odor or bacteria. The resultant product may be mixed with beeswax and scented prior to being poured and cooled in containers.

### I. CLAIM OF PRIORITY

This application is a continuation patent application of, and claims priority from, U.S. Provisional Patent Application Ser. No. 62/042,048, filed on Aug. 26, 2014, which is incorporated by reference herein in its entirety for all purposes.

### II. FIELD OF THE DISCLOSURE

The disclosure relates to personal hygiene products, such as deodorant.

### III. BACKGROUND

Chemicals additives found in many deodorants are absorbed through the skin. Some of the additives have been linked to different ailments, including cancer. Consequently, some cancer survivors are instructed not to wear antiperspirants and deodorants that contain aluminum and other additives. In an increasingly health conscious society, there is a need for a safe alternative for treating body odor.

### IV. SUMMARY OF THE DISCLOSURE

A particular embodiment includes manufacturing a substance to treat body odor or bacteria by providing a supply of ozone and providing unsaturated fat. The ozone is introduced into the unsaturated fat to create an ozonated unsaturated fat substance to treat odor or bacteria.

According to an embodiment, the ozonated unsaturated fat substance is mixed with a second substance. The second substance may have a higher melting point than the ozonated unsaturated fat substance. The second substance may include beeswax.

An essential oil or fragrance may be mixed into the ozonated unsaturated fat substance. The supply of ozone may be created. The unsaturated fat may be melted. The unsaturated fat may include at least one of: olive oil, coconut oil, shea butter, hobo oil, and wax ester. The ozonated unsaturated fat substance may be an ozonide. The reaction may additionally produce at least one of a: hydroperoxide, an aldehyde, a peroxide, a diperoxide, and a polyperoxide.

According to another particular embodiment, an apparatus includes a container and an ozonated unsaturated fat substance included in the container. The ozonated unsaturated fat substance may be exposed to ozone to create a product resistant to odor and bacteria.

These and other advantages and features that characterize embodiments are set forth in the claims annexed hereto and forming a further part hereof. However, for a better understanding of the invention, and of the advantages and objectives attained through its use, reference should be made to the Drawings and to the accompanying descriptive matter in which there are described exemplary embodiments.

### V. BRIEF DESCRIPTION OF THE DRAWINGS

FIG. 1 is a block diagram of an embodiment of a system configured to produce an ozone enhanced product to retard odor and bacteria; and

FIG. 2 is flowchart of processes executable by an embodiment similar to FIG. 1 to produce an ozone enhanced product for combating odor and bacteria.

### VI. DETAILED DESCRIPTION

An embodiment may enhance a substance with ozone such that the use of that enhanced substance on skin prevents, removes, improves, or otherwise treats body odor. In a specific embodiment, unsaturated fatty acids receive ozone gas. The resultant ozone enhanced substance is used as deodorizers for the body. For example, the substance is applied anywhere on the body for purpose of eliminating or oxidizing bacteria, especially the anaerobic bacteria responsible for body odor.

An embodiment includes ozonating (i.e., ozonizing, infusing oxygen/ozone, or bubbling ozone gas through) an unsaturated fatty acid. Illustrative unsaturated fats include olive oil, coconut oil, shea butter, hobo oil, and wax ester, among others. The process creates ozonides of unsaturated fatty acids, or oxygen-rich unsaturated fatty acids, or other compounds, oxygen related or otherwise.

The resultant substance oxidizes the cell wall of anaerobic bacteria that causes body odor. More particularly, an extra oxygen molecule of the ozone is attracted to cell walls that are weakened by disease (e.g., viral, fungal, bacterial). Their collision may destroy sick cells while leaving healthy cells alone.

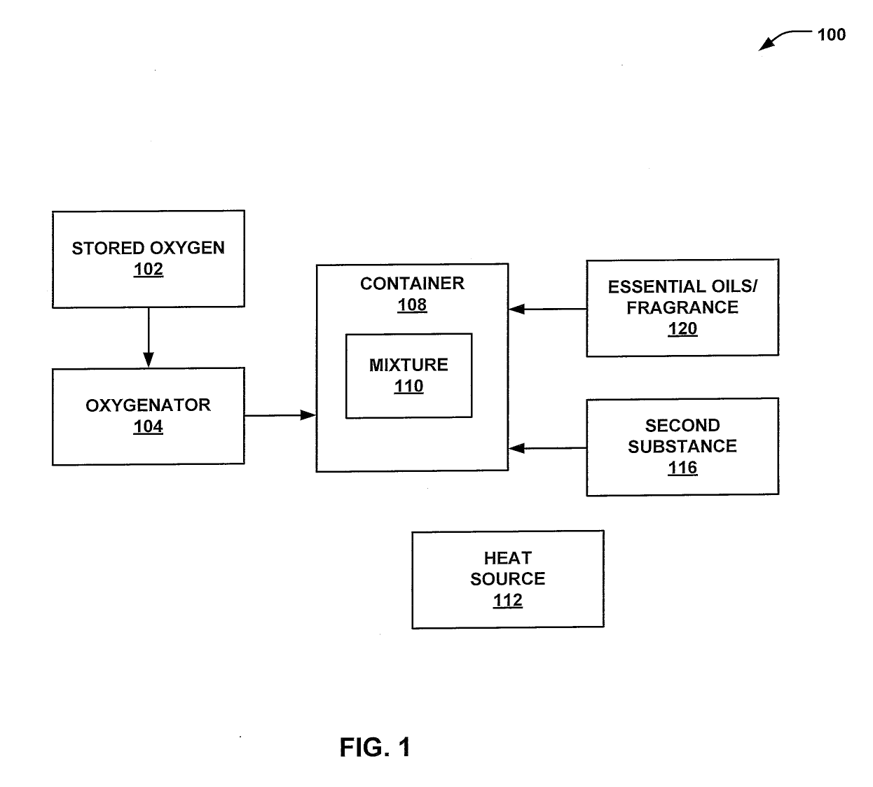

FIG. 1 shows a system 100 for producing a substance that inhibits odor and bacteria. The system 100 includes stored oxygen 102 coupled to an oxygenator 104. The oxygenator 104 may produce ozone 106 that is supplied to a container 108 that initially includes an unsaturated fat. A heat source 112 may regulate the temperature of the container 108. A second substance 116, such as bees wax, may be heated and supplied to the container 108 of oxonated unsaturated fat (i.e., unsaturated fatty acid). Where desired, essential oils 120 may be added to the mixture 110.

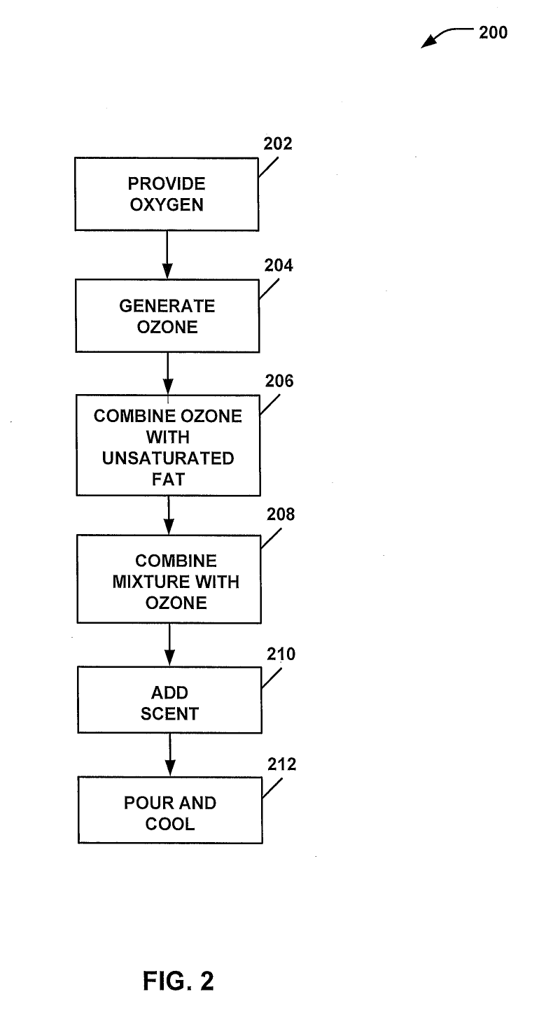

The flowchart of FIG. 2 shows the processes of an embodiment that may be executed by the illustrative system 100 of FIG. 1. At 202, oxygen is provided, such as may be produced by an oxygen concentrator. Pure oxygen may be used to achieve a higher concentration (purer oxygen may produce purer ozone) as ozone is generated at 204. The ozone of an embodiment may be generated by corona discharge or using ultraviolet plates. The rate at which the oxygen may be provided may range from 1 to 1.5 liters per minute to reduce splashing and bubbling, and to increase the concentration. For instance, a six percent ozone concentration may be achieved by weight. The range may further reduce the time spent producing the finished product.

The ozone may be introduced to an unsaturated fat at 206. For instance, an oxygenator may feed ozone gas into a bottom of a container of unsaturated fat. Examples of unsaturated fats include shea butter, coconut oil, wax ester, hobo oil, and olive oil, among others. Unsaturated fats may absorb a reactive oxygen atom of the introduced ozone. Another embodiment may use a substance which, like unsaturated fat, reacts with ozone.

Where desired, the unsaturated fat may be melted. For instance, shea butter may be melted using a modified wax melter bowl at a range of between 95 and 160 degrees Fahrenheit. Melting may increase the efficiency of absorption. The ozone gas may be bubbled through the unsaturated fat for a range of 18-26 hours (optimally 23 hours) for sufficient infusion of ozone into unsaturated fat. This rate may maximize exposure to the ozone. Ozonides of unsaturated fatty acids are thus obtained. The reaction may also produce hydroperoxides, aldehydes, peroxides, diperoxides, and polyperoxides, all of which may be useful in eliminating bacteria on the skin.

The ozonated unsaturated fat may be mixed at 208 with beeswax, or another substance that may provide hardness and keeps the resultant product solid at room temperature. Ideally, the substance provides for the safety and wellness of skin, in addition to raising the melting point. Beeswax advantageously facilitates a subtle melting of a top layer of the mixed product in response to body heat.

More particularly at 208, the ozonated unsaturated fat may be maintained at a temperature of around 110 degrees Fahrenheit to maintain a liquid state. Beeswax pastilles may be melted at around 350 degrees Fahrenheit for around 30 minutes and are added to the ozonated unsaturated fat. One illustrative mixture may result in 80% ozonated unsaturated fat and 20% beeswax.

Essential oils and/or fragrance, such as jasmine, lemon grass, or rosemary, may be added at 210. The mixed solution may be poured at 212 into deodorant or other containers and allowed to cool at room temperature to avoid cracking.

The resultant ozone unsaturated fat may be configured to kill and prevent the growth of bacteria, while deodorizing. The oxygen in the product may further acts as a preservative without chemical additives.

Those skilled in the art may make numerous uses and modifications of and departures from the specific apparatus and techniques disclosed herein without departing from the inventive concepts. Consequently, the disclosed embodiments should be construed as embracing each and every novel feature and novel combination of features present in or possessed by the apparatus and techniques disclosed herein and limited only by the scope of the appended claims, and equivalents thereof.

1. A method of manufacturing a substance to treat body odor or bacteria, the method comprising:
providing a supply of ozone; and
providing unsaturated fat; and
introducing the ozone into the unsaturated fat to create an ozonated unsaturated fat substance to treat odor or bacteria.

2. The method of claim 1, further comprising mixing the ozonated unsaturated fat substance with a second substance.

3. The method of claim 2, wherein the second substance has a higher melting point than the ozonated unsaturated fat substance.

4. The method of claim 2, wherein the second substance is beeswax.

5. The method of claim 1, further comprising adding an essential oil or fragrance into the ozonated unsaturated fat substance.

6. The method of claim 1, further comprising creating the supply of ozone.

7. The method of claim 1, further comprising melting the unsaturated fat.

8. The method of claim 1, wherein the unsaturated fat includes at least one of: olive oil, coconut oil, shea butter, hobo oil, and wax ester.

9. The method of claim 1, wherein the ozonated unsaturated fat substance is an ozonide.

10. The method of claim 1, further comprising at least one of a: hydroperoxide, an aldehyde, a peroxide, a diperoxide, and a polyperoxide.

11. An apparatus comprising:
a container; and
an ozonated unsaturated fat substance included in the container, wherein the ozonated unsaturated fat substance is exposed to ozone to create a product resistant to odor and bacteria.
